(19) United States
(12) Patent Application Publication
(10) Pub. No.: US 2006/0188585 A1
(43) Pub. Date: Aug. 24, 2006

(54) NERIUM OLEANDER EXTRACT

(76) Inventors: Aleksandr Panosyan, Yerevan (AM); Saleh Mohammed Al-Mukarish, Jeddah (SA)

Correspondence Address: Joseph Hyosuk Kim, Ph. D.;JHK Law, P.O. Box 1078, La Canada, CA, 91012-1078, US

(21) Appl. No.: 10/326,975

(22) Filed: Dec. 24, 2002

Related U.S. Application Data
(63) Continuation-in-part of application No. PCT/CY01/00002, filed on Jun. 18, 2001.

(51) Int. Cl. A61K 36/484; A61K 36/185

(52) U.S. Cl. 424/725; 424/757

(57) ABSTRACT

The present invention is directed to γ-Interferon-inducing activity of Nerium oleander, special extract, its ingredients, and compositions containing fixed combinations of the Nerium oleazide, and Glycyrrhiza glabra special extracts, as well as to a method of preparation of these extracts using a substance's characteristics infonnation transmission technique.

### CROSS-REFERENCE To RELATED APPLICATIONS

This application claims the benefit of PCT/CY01/00002, filed Jun. 18, 2001, which disclosure is herein incorporated by reference.

### BACKGROUND OF THE INVENTION

1. Field of the Invention

This invention relates to the field of interferon (ifn) inducers—a special group of potential antiviral compounds, particularly to Nerium oleander special extract, its ingredients and compositions containing fixed combinations of the Nerium oleander and Glycyrrhiza glabra extracts, as well as to a method of preparation of the extracts using a substance's characteristics information transmission device.

2. General Background and State of the Art

The ability of IFN-s to confer an antiviral state on cells is their defining activity as well as the fundamental property. IFNs are essential for the survival of higher vertebrates because they provide an early line of defense against viral infections—hours to days before immune responses. This vital role has been demonstrated by the exquisite sensitivity to virus infections of mice lacking both IFN α/βand γ receptors. Multiple pathways have involved to combat different types of viruses and the various compensatory defense mechanisms that different viruses have involved.

Antiviral mechanisms of interferon action include dsRNA-dependent protein kinase (PKR), the 2-5A Synthases, and the Mx proteins pathways. PKR pathway mediates signal transduction, inhibition of protein synthesis and transcriptional control, 2-5A system pathway—RNA cleavage, and Mx proteins interfere with viral replication, impairing the growth of influenza and other negative-strand RNA viruses at the level of viral transcription and at other steps

Any stage in virus replication appears to be inhibited by IFNs, including entry and/or uncoating (simian virus 40, retroviruses), transcription (influenza virus, vesicular stomatitis virus), RNA stability (picomaviruses), initiation of translation (reoviruses, adenovirus, vaccina), maturation, and assembly and release (retroviruses). Along with antiviral activity IFNs inhibit cell growth and control apoptosis.

Effects of γ-IFN on the immune system includes the ability to induce the expression of MHC class II proteins in a wide variety of different cell types, and thereby to promote the development of CD4+T-cell response. It further includes generation of activated macrophlages, a key effector cell population in innate and adaptive immune responses involved in killing microbial targets. γ-IFN also regulates humoral immunity by regulating the development of specific T helper cell subsets, or directly at the level of B cells and their functions—development of proliferation, immunoglobulin secretion and IG heavy-chain switching.

IFN inducers represent a special group of potential antiviral compounds. The main requirements for them are (1) high IFN—inducing activity, (2) absence od side effects, (3) wide range of antimicrobial activity, (4) broad therapeutic security and, (5) good solubility in water and biologic fluids. IFN inducers may be used against very different infections and conditions. IFN inducers stimulate IFN production in different cells and organs, and that determine the strategy for their application in hepatitis B and C, influenza, rhinioviral and enteroviral infections, encephalitis, rabies, etc.

It has been demonstrated that glycyrrhizin, an active principle of Glycyrrhiza glabra extracts, induces interferon formation. Antiviral activity of glycyrrhizin was observed against vaccinia, herpes simplex I, Newcastle disease, vesicular stomatitis and influenza A viruses.

It has been shown in several publications that extracts of Nerium oleander, leaves, well known as a very poisonous plant due to presence of cardiac glycosides, have antitumor, immunomodulating and antibacterial activity. The results of these studies are very controversial both about efficacy and the main constituents responsible for these effects.

As is described in U.S. Pat. No. 5,135,745 “Extracts of Nerium species, methods of preparation, and use therefore”, the water extract of Nerium oleander, is useful in ameliorating cell-proliferative disease in animals. The patent discloses a polysaccharide enriched extract of Nerium species containing an immunologically active polysaccharide useful in treating cell-proliferative disease in mammals, wherein the active polysaccharide comprises acidic homo-poly-galacturonans or arabino-galacturonans. The patent also discloses a method of preparation of Nerium species containing an immunologically active polysaccharide useful in ameliorating cell-proliferative disease in mammals, by boiling of plant material in inorganic solvent for several hours to obtain density about 1010. And the patent further discloses a method of ameliorating cell-proliferative diseases (malignancy, adenocarcinoma, psoriasis) by administrating (parenterally, subcutaneously, intramuscularly, intraperitoneally, intracavity, intravenously, transdermal, nasopharyngeally, or mucosal absorption) of a polysacchlaride enriched extract of Nerium species extracts.

However, it has never been scientifically demonstrated that extracts of Nerium oleander and its components could have IFN-inducing and/or antiviral activity. Nerium species have never been used in combination with other herbal drugs, particularly with Glycyrrhiza extract for treatment of any diseases, The described method of preparation of the Nerium extracts in the prior art does not include treatment with a substance's characteristics information transmission technique for the purpose of potentiation of the activity of the final product.

Hardly surprising, viruses fight back, not only against host defenses in general, but also against the IFN system in particular, both through novel mechanisms and by subverting host systems through the synthesis of novel proteins and proteins that mimic and thus interfere with host systems γ-IFN is generated mainly in T cells.

Many factors are involved in the activation and suppression of T cells. Consequently, there might be multiple modes of INF induction by T cells. Increasingly, research shows that fixed combination of herbs have greater-than-expected medicinal benefit for the treatment of viral diseases, due to the combination of constituents, which have synergistic effect and act on different molecular targets. In some cases, the medicinal value of the herb may be entirely due to the combination of substances and cannot be reproduced by one or two “active” constituents alone. Complex combination of several plants is a general approach of traditional medicine, particularly of oriental medicine such as Ayurveda and Unani in India, Kampo in China and Japan.

Following this concept the present application discloses the efficacy of a fixed combination of two plant extracts standardized for their active ingredients: Glycyrrhizin (Glycyrrhiza glabra L. ) and polysaccharides (Nerium oleander L.). The application further discloses a potentiating effect of a substance's characteristics information transmission technique [Gotovski Yu et al . . . , Patents of Russia 2,070,405, 2,070,406, 2,065,297, 1996] on the IFN-inducing activity of the extracts. In addition the application discloses in vitro experiments on whole blood cell culture (IFN-inducing activity) and on human carcinoma squamous HEp-2 cells infected by mice encephala-myocarditis virus (virucide effect).

### SUMMARY OF THE INVENTION

The present invention is directed to a variety of embodiments. In one aspect, the invention is directed to a virucidal composition comprising a γ-IFN-induciing effective amount of Nerium oleander extract, which extract is processed with straight transmission of γ-IFN infonnation characteristics and transmission of information characteristics of human organism specific response viruses by a substances' characteristics information transmission device.

The virus may be any virus that is susceptible to combat by the presence of interferon. In particular, the virus may be a Hepatitis A, B, C, or D virus, or HIV virus, or a virus that causes measles.

In making the above composition, the extract may be obtained from any part of the plant. In particular, the extract may be made from chopped leaves, stems and/or flowers.

In one aspect of the invention, the γ-IFN information characteristics and transmission device may be Transfer-P.

In still another aspect of the invention, the invention is directed to a method of preparing a virucidal composition comprising a γ-IFN-inducing effective amount of Nerium oleander extract, comprising exposing an extract of N. oleander to a straight transmission of γ-IFN information characteristics and transmission of information characteristics of human organism specific response on viruses by substances' characteristics information transmission device. In this method, in particulare, the virus may be Hepatitis A, B, C, D, HIV or measles causing virus. And further in particular, Transfer-P device may be used to potentiate the solution to form an effective antiviral composition.

In another embodiment of the invention, the invention is directed to a virucidal composition comprising a γ-IFN-inducing effective amount of Nerium oleander and Glycyrrhiza glabra extracts. In this composition, the N. oleander, extract may be obtained from chopped leaves, stems and/or flower, and G. glabra extract may be obtained from roots, while other parts of the plants may also be sources for the extracts.

The above composition also may be comprised of extracts that have been processed with straight transmission of γ-IFN infonmation characteristics and transmission of information characteristics of human organism specific response viruses by a substances' characteristics infonnation transmission device. In particular, the virus may be Hepatitis A, B, C, D, HIV or measles causing viruses. In this composition the γ-IFN infoimation characteristics and transmission device may be Transfer-P.

In yet another aspect of the invention, the invention is directed to a method of preparing the composition, comprising exposing an extract of N. oleander, and/or Glycyrrhiza glabra extract to a straight transmission of γ-IFN infonnation characteristics and transmission of infonnation characteristics of human organism specific response on viruses by substances' characteristics information transmission device. In particular, the virus may be Hepatitis A, B, C, D, HIV or measles causing virus, and device in particular may be Transfer-P. The invention is also directed to a method for treating viral infection, comprising administering to a person in need thereof any of the compositions described above. In particular the viral infection may be caused by Hepatitis A, B, C, D, HIV or measles virus.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 shows HPLC of Nerium oleander, special extract. Details are shown on the figure. Box inset shows polysaccharides MW calibration curve.

FIG. 2 shows TMS-ethers of hydrolysates of NO polysaccharides fractions PS-I, PS-II and PS-III. Peaks identified are as follows: 1-trihydroxybutiric acid; 2-ribose; 3-arabinose; 4-xylose; 5-lyxose; 6-mannose; 7-talose; 8-glucitol; 9-glucose; 10-galacturonic acid; 11-not identified Compound X. Inset box shows—mass-spectra of Compound X—TMS ether.

FIG. 3 shows a chromatogram of the FC-NO:GG extract—TMS derivates before (A) and after (B) hydrolysis (AS-1):Identification of the peaks in Total Ion Current mode.

FIG. 4 shows effect of NOE, GGE, NOSE, FC-NO:GG, FC-NO:GG-SE, NO-PS-I, NO-PS-II, NO-PS-III on γ-IFN (pg/ml) induction in human whole blood cell culture in vitro.

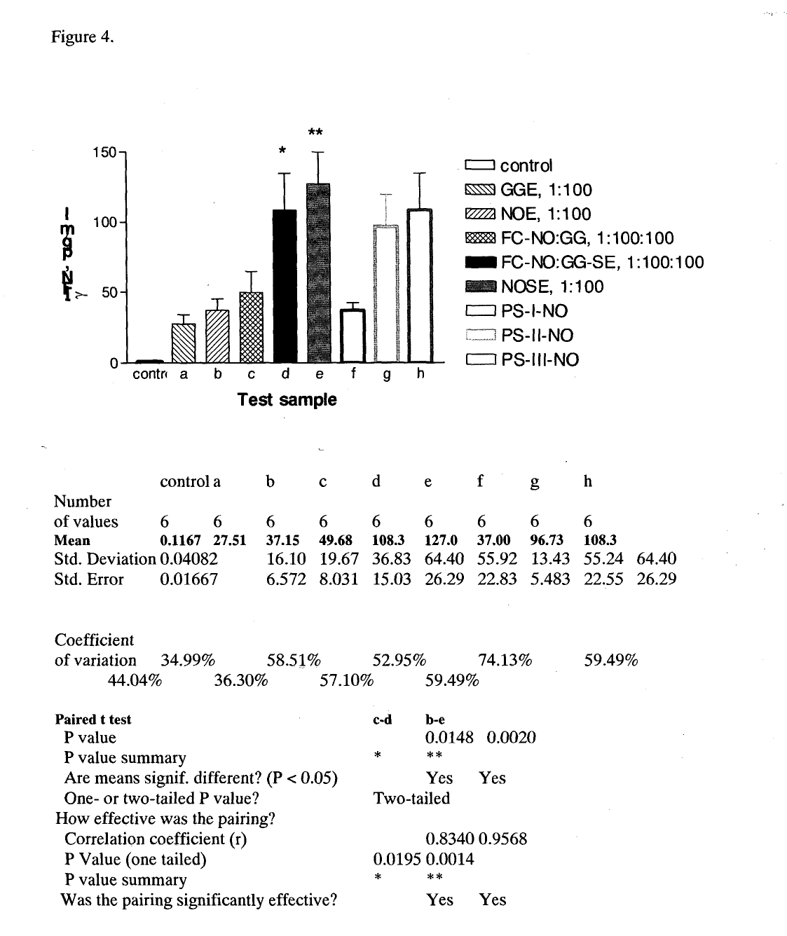

### DETAILED DESCRIPTION OF TIIE PREFERRED EMBODIMENTS

In the present application, “a” and “an” are used to refer to both single and a plurality of objects.

The present invention relates to new γ-Interferon-inducing agents, more particularly to the Nerium oleander extract, its active ingredients and its fixed combinations with Glycyrrhiza glabra extracts, and to the method of their preparation using a substance's characteristics infonnation transmission technique for potentiation of the activity of extracts and uses therefore as potential antiviral drugs.

In general, to obtain Nerium species extracts, water extraction of the air dried leaves, flowers and stems at high temperature for several hours (enough for decomposition of toxic cardiac glycosides) is used in accordance with U.S. Pat. No. 5,135,745, which disclosed the extract itself, as well the use of this extract in ameliorating cell-proliferative diseases (malignancy, adenocarcinoma, psoriasis) by administrating (parenterally, subcutaneously, intramuscularly, intraperitoneally, intracavity, intravenously, transdennal, nasopharyngeally, or mucosal absorption).

The present invention concerns a new method of preparation of a special extract of Nerium oleander leaves, which exhibits new property, particularly IFN inducing activity. This special extract is significantly different from those mentioned in US patent extract prepared without any treatment with the substance's information transmission technique, particularly in vitro experiments where IFN-inducing activity is evaluated.

Active ingredients of these extracts are shown to be 50 to 500 KDa polysaccharides containing trihydroxybutyric acid, ribose, arabinose, xylose, lyxose, mannose, talose, glucitol, glucose and galacturonic acid.

Another object of theinvention is the fixed combination of Nerium oleander leaves and Glycyrrhiza glabra roots, potentially useful in treating viral disease in mammals, which exhibits γ-IFN-inducing and virucide activity in in vitro experiments on whole blood cell culture (IFN-inducing activity) and on human carcinoma squamous HEp-2 cells infected by the mice encephala-myocarditis virus (virucide effect). These fixed combination of Nerium oleander, and Glycyrrhiza glabra extracts (FC-NO:GG) are more active than each component separately (NOE and GGE). Fixed combination of special extracts (FC-NO:GG-SE) obtained by the treatment of substance's characteristics infonnation transmission device is significantly more active than samples FC-NO:GG (without such a treatment).

In order to determine whether patients with viral diseases, namely with hepatitis B or C, have benefit from therapy based upon the extract of the invention, four patients diagnosed as acute hepatitis B and C were treated with a standardized fixed combination of special extracts (FC-NO:GG-SE). The liquid extract, standardized for the content of polysaccharides (0.7-1.1 mg/ml), glycyrrlhizin (0.85-1.15 mg/ml) and substance's characteristics information, was taken orally in a daily dose of 1-1.5 ml×2 for 1.5-6 months. At the end of the treatment the laboratory tests for HbsAg (n=2) and HCV PCR (n=2) were negative in all four cases.

The present invention is not to be limited in scope by the specific embodiments described herein. Indeed, various modifications of the invention in addition to those described herein will become apparent to theose skilled in the art from the foregoing description and accompanying figures. Such modifications are intended to fall within the scope of the appended claims. The following examples are offered by way of illustration of the present invention, and not by way of limitation.

### EXAMPLES

### Example 1

### Preparation of Nerium oleander L. Extract (NOE)

Add about 10 g of air dried, sliced into pieces leaves, branches and flowers of Nerium oleander to 100 ml of distilled water in 200 ml of round-bottom flask, heat it up to 100° C. and keep boiling for 3 h. During boiling, add distilled water to maintain a constant water level. After boiling, bring the temperature of the mixture to 20-25° C., filter using a coarse filter in order to separate from plant material and remove from particulate matters. Bring filtrate to the volume of 100 ml with distilled water and subject it to sterile filtration into sterile flask using Millipore filters with pore size 0.45 μM and 0.22 μM consequently.

Specification:

Total saccharides content—3.9-4.1 mg/ml, total polysaccharides content—1.4-1.6 mg/ml, dry residue—10-12 mg/ml.

### Example 2

### Preparation of Nerium oleander L. special extract (NOSE)

Add about 10 g of air dried, sliced into pieces leaves, branches and flowers of Nerium oleander to 100 ml of distilled water in 200 ml of round-bottom flask, heat it up to 100° C. and keep boiling for 3 h. During boiling, add distilled water to maintain a constant water level. After boiling, bring the temperature of the mixture to 20-25° C., filter using a coarse filter in order to separate from plant material and remove from particulate matters. Bring filtrate to the volume of 100 ml with distilled water and subject it to sterile filtration into sterile flask using Millipore filters with pore size 0.45 μM and 0.22 μM consequently. On the filling line after sterile filtration fix active electrodes of apparatus for the substance's characteristics information transmission “Transfer-P” (Imedis-BRT Ltd., Moscow). The apparatus is set up on the straight transmission of γ-IFN information characteristics and transmission of information characteristics of human organism specific response on certain groups of viruses (Hepatitis A, HIV, measles) by “Imedis Bioresonance” software equipped with Pauly & Shmidt's “Substances' Bio-resonance Database” (Imedis-BRT Ltd., Moscow). Store solution at 4° C. for one year.

Transfer-P devices are sold through Imedis-BRT Ltd., Moscow. Transfer-P transfers energy-informative transfer (with inversion if needed) of properties of homocopathic preparations onto carriers with ability to change potency. This device does not require computer and cannot be connected to computer. Without limiting the device to any specific dimensions, Transfer-P device is enclosed with tiny plastic chassis (157×95×40 mm). Device is provided with all required wires. The weight of the device is only 0.15 kg. Transfer-P device has no power supply.

Transfer-P device allows one to carry out an energy-informative transfer of properties of substances onto neutral carriers, directly, or with inversion; to change potencies of homeopathic remedies and therefore to make individual selection of potencies using medicamental testing by R. Voll; to carry out remote contactless influence of homeopathic remedics to a patient and check their effect; to make a transfer on to large volumes of liquid (water, physiological solution, alcohol etc.).

Specification: Total saccharides content—3.9-4.1 mg/ml, total polysaccharides content—1.4-1.6 mg/ml, dry residue—10-12 mg/ml.

### Example 3

### Isolation of Nerium oleander polysaccharides (NOPS)

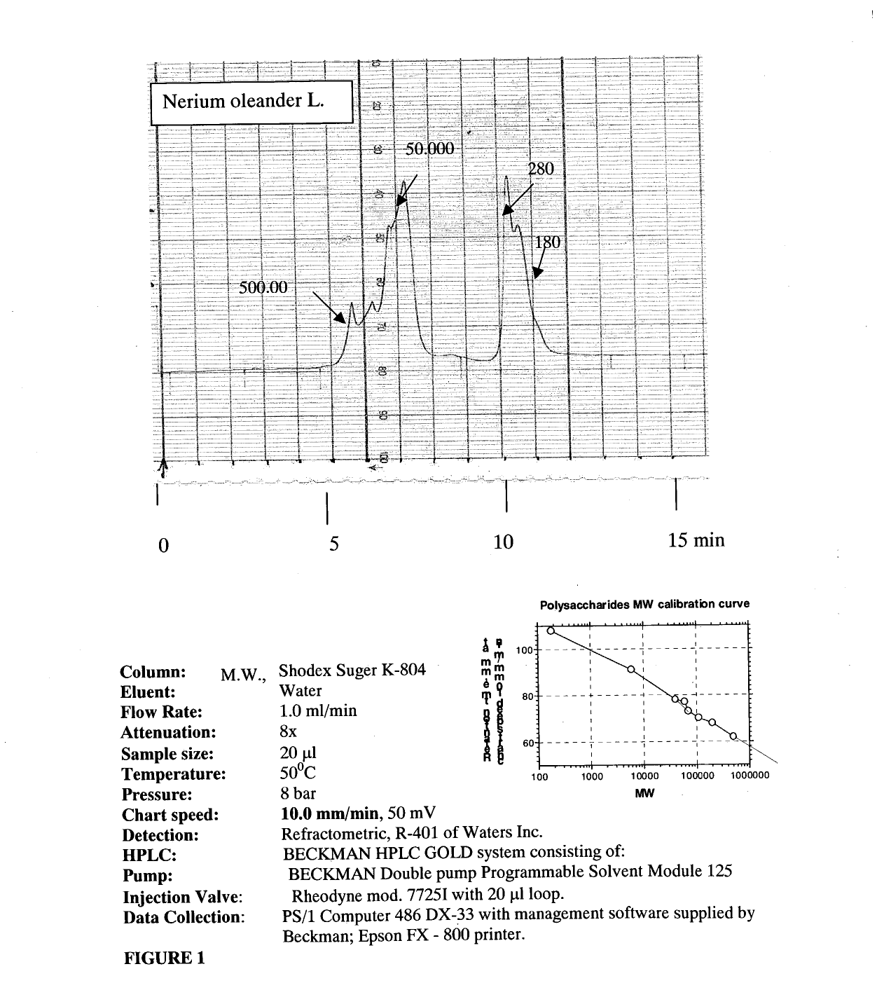

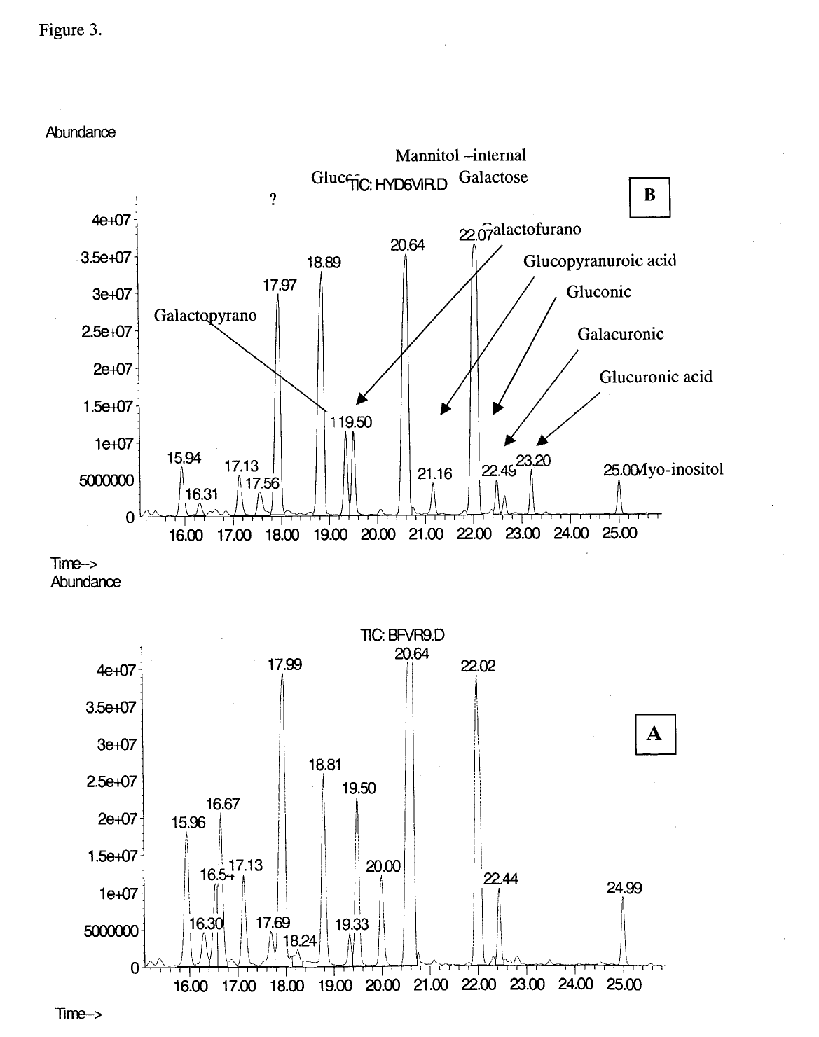

Apply 20 μl of NOE to Shodex Suger lonpak K-804 ion-exchange resin gel of ed styrene-divinylbenzene copolymer HPLC column (Showa Denko K.K., Japan) and column with water to obtain PS fractions I-III, FIG. 1. These fractions were further characterized for their monosaccharides composition by GC-MS of their TMS-derivatives, FIG 3.

### Example 4

### Preparation of Glycyririziza glabra L. Extract (GGE)

Glycyrrhiza glabra L. extract (GGE) is prepared as described above for NOE Example 1).

Specification:

Glycyrrhizin content—2.0-2.5 mg/ml, total saccharides content—4.3-4.5 mg/ml, total polysaccharides content—2.8-3.0 mg/ml, dry residue—12-14 mg/ml.

### Example 5

### Preparation of the fixed combination of the extracts of Nerium oleander and Glycyrrhiza glabra (FC-NO:GG-SE).

Add about 5 g of air dried, sliced into pieces leaves, branches and flowers of Nerium oleander and 5 g of air dried, sliced into pieces roots of Glycyrrhiza glabra to 100 ml of disilled water in 200 ml of round-bottom flask, heat it up to 100° C. and keep boiling for 3 h. During boiling, add distilled water to maintain a constant water level. After boiling, bring the temperature of the mixture to 20-25 ° C., filter using a coarse filter in order to separate from plant material and remove from particulate matters. Bring filtrate to the volume of 100 ml with distilled water and subject it to sterile filtration into sterile flask using Millipore filters with pore size 0.45 μM and 0.22 μM consequently. On the filling line after sterile filtration fix active electrodes of apparatus for the substance's characteristics information transmission “Transfer-P” (Imedis-BRT Ltd., Moscow). The apparatus is set up on the straight transmission of γ-IFN information characteristics and transmission of information characteristics of human organism specific response on certain groups of viruses (Hepatitis A, B, C, D, measles) “Imedis Bioresonance” software equipped with Pauly & Shmidt“s “Substances' Bio-resonance Database” (Imedis-BRT Ltd., Moscow).

Specification:

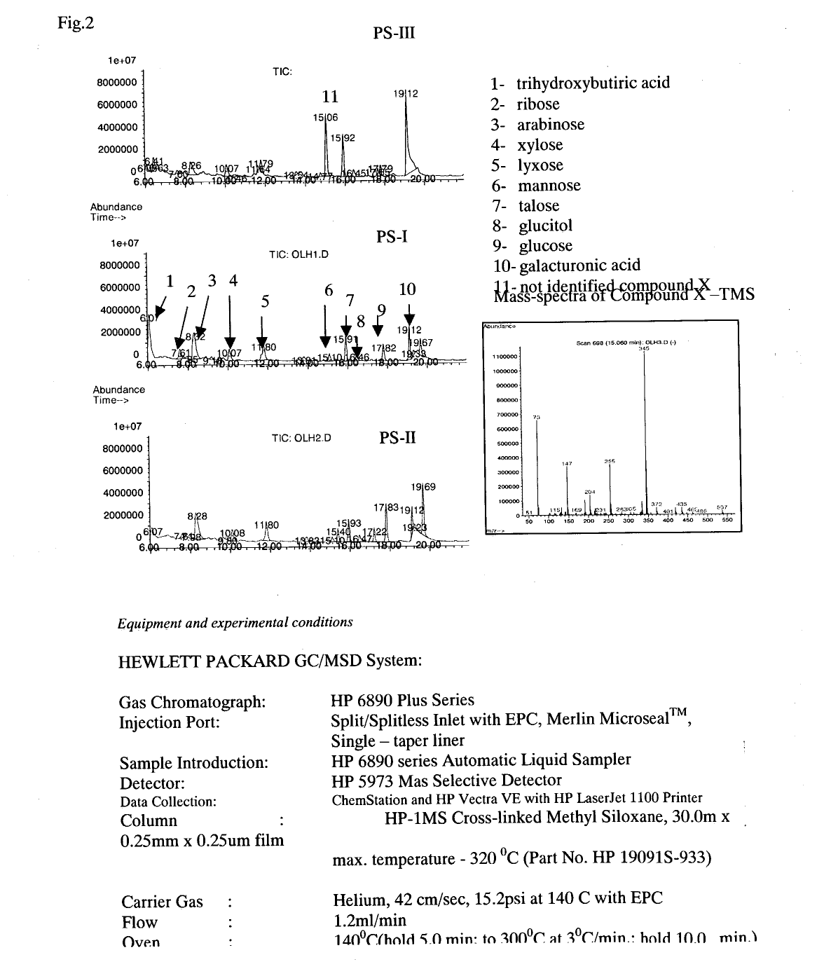

Total saccharides content—5.0-5.4 mg/ml, total polysaccharides content—1.4-1.6 mg/ml, Glycyrrhizin—1.15 mg/ml, dry residue—13-15 mg/ml. Free and bound monosaccharides were characterized by GC-MS of their TMS-derivatives, FIG 2.

### Example 6

### IFN-γ inducing activity of the extracts

Test Samples:

Concentrations of the extracts and their ingredients in stock solution used for further dilutions of test samples were:

(a) 10.4 mg/ml of Nerium oleander extract (NOE) with 1.5 mg/ml of Nerium oleander polysaccharides (NOPS);

(b) 13.9 mg/ml of Glycyrrhiza glabra extract (GGE) with 2.9 mg/ml of Glycyrrhiza glabra PS and 2.3 mg/ml of Glycyrrhizin;

(c) 13.9 mg/ml of fixed combination (FC) of NOE:GGE with 1.2 mg/ml of PS and 1.15 mg/ml of Glycyrrhizin;

(d) 13.9 mg/ml of fixed combination Nerium oleander and Glycyrrhiza glabra special extract FC-NO:GG-SE with 1.2 mg/ml of PS and 1.15 mg/ml of Glycyrrhizin;

(e) 10.4 mg/ml of Nerium oleander special extract (NOSE) with 1.5 mg/ml of NOPS;

(f) 0.2 mg/ml of NO-PS-I;

(g) 0.2 mg/ml of NO-PS-II; and

(h) 1.0 mg/ml of NO-PS-III

Then solutions (a), (b), (e), (f) and (g) were diluted with water in ten times (1:9), while solutions (c), (d) in five times (1:4), to obtain dilution (1:10) with concentrations of Glycyrrhizin and PS equal to that in all other samples (a)-(g). From these solutions other dilutions (1:100, 1:1000 and 1:10000) of test samples were prepared.

Final concentrations of the extracts and their ingredients in incubation media were ten times less than in test samples since one volume of test sample is added to nine volumes of incubation media.

Incubation procedure

Effect of extracts and their components on INF-γ production in whole blood cell culture was studied as described by Wang et al., 2000. Whole blood of healthy donors (n=6) was collected into 4 ml sterile heparinized (lithium heparin) Vacutainer tubes (Vacuete, Greiner). 100 μl of blood samples were added to each well of 12-well tissue culture plate (Falcon), containing 800 μl cultivation media (RPMI- 1640 medium containing 2 ml L-glutamine, 1 mM sodium pyruvate, 100 IU penicillin and streptomycin and 10% fetal calf serum) and 100 μl test solutions with known concentrations of active ingredients, glycyrrhizin and polysaccharides (PS). Cells were cultivated for 72 hours in a humidifier atmosphere, containing 6% CO₂. and centrifuged in JOUAN BR4 centrifuge rotor with 12-well plate adapters at 3000 rpm for 15 min. Cell-free culture supernatants were collected and stored at—80° C. until use.

IFN-γ Assay

IFN-γ content in cell culture supernatants was determined using commercially available human Interferon-γ TiterZyme²⁰⁰ EIA kits (Assay Designs, Inc. MI, USA) as described in instruction sheet. IFN-γ Standards calibration curve was prepared and concentrations of IFN-γ were automatically calculated using optical density data at 450 nm obtained by multichannel microplate-reader PR-2100 (Sanofi, Pasteur). All measurements were performed in duplicate, means and standard deviations were recorded.

Results

All tested samples were active in dilutions 1:100 (actually in final dilutions 1:1000), FIG. 3. That corresponds to concentrations of extracts from 10 to 30 μg/ml, polysaccharides—from 1.5 to 3 μg/ml, and glycyrthizin—2.2 μg/ml (2.7 μM). At lower concentrations they were inactive, and in higher—cytotoxic. FIG. 3 shows that Nerium oleander extracts (NOE and NOSE) stimulates γ-IFN production, and polysaccharides I-III are active ingredients of these extracts. FIG. 3 also shows that fixed combination of Nerium oleander and Glycyrrhiza glabra extracts (FC-NO:GG) is more active than each component separately (NOE and GGE). In addition, FIG. 3 shows that usage of substance's characteristics information transmission technique significantly potentates the IFN induction in whole blood in vitro test. Test samples of NOSE and FC-NO:GG-SE obtained by the treatment of the substance's characteristics information transmission device are significantly more active than samples NOE and FC-NO:GG (without such a treatment); all these samples have the same concentrations of active ingredients.

### Example 7

### Antiviral activity of FC-NO:GG extract

Materials and Methods

Various methods are available for in vitro and in vivo antiviral screening of plant materials (Vlietinck and Vanden Berghe, 1991). In our study we used methods earlier described for encephalitis virus (Fokina et al., 1991).

The followinig materials were used:

Eagle's growth medium with Earl's solution containing 10% fetal bovine serum (FBS) purchased from the Chumakov's Institute of Poliomyelitis and Viral Encephalitis, Moscow, Russia.

0.25% Trypsin solution. Chumakov's Institute of Poliomyelitis and Viral Encephalitis, Moscow, Russia.

0.02% Versenate Solution. Chumakov's Institute of Poliomyelitis and Viral Encephalitis, Moscow, Russia

Fetal bovine serum (FBS). Chumakov's Institute of Poliomyelitis and Viral Encephalitis, Moscow, Russia. Each batch of the FBS is inactivated for 30 min at 56° C. before use.

Maintaining medium—Eagle's minimal medium containing 2% fetal serum.

Goriaew's hemocytometer (Krasnogvardeec Company, Sankt-Petersburg, Russia).

50 ml and 250 ml vials (Costar Co.)

0.02-1 ml adjustable automatic single channel ant multi-channel samplers,. (Labsystems, Finland).

96 well MaxiSorp Nunc microplates—COSTAR.

0.025 ml multi-channel sampler with dosing tips.

0.025 ml and 0.1 ml sampler.

Thermostat with CO₂ supply.

Sterile containers and pipets for dilution.

Virus suspension with known concentration

Methods:

Passage and maintenance of human carcinoma squamous cell HEp-2 (Caucasian, larynx) and mouse embryo cell NIH 3T3 (NIH Swiss), cultures.

Re-sowing of the cells

Pour out the growth media from the vial containing the cell culture;

Add 0.25% Trypsin solution (or equal volumes of 0.25% Trypsin solution and 1:5000 Solutio Versenate) into the vial. 0.5 ml of the solution is enough for the vial with 25 cm² surface. Distribute it equally on the surface of cells gently shaking of the vial;

Keep the vial was stored at 36° C., unless all cells are separated from the growth surface (control by microscopic examination)

Re-suspended the cells in the growth medium (4.5 ml per vial with 25 cm² surface), which is neglecting the trypsin action. Suck in the suspension several times through a thin sampler tip to disintegrate the cells aggregations;

Dilute the cell suspension in the growth medium to required concentration, or dilute it in ratio 1:2 v/v.;

Sow the cells suspension in vials, close tightly and place in thermostat at 36° C.

Incubate cell culture for 48-72 hours to obtain monolayer, then change the growth medium for maintenance media. Interweave the cell culture every 5-7 days.

Cell count

The cell count can be exactly counted in the suspension by a hemocytometer. Thoroughly disperse cells by recurrent suction through a thin dropper before the counting. Dilute 0.1 ml of cell suspension with 0.9 ml of 0.1% Trypan Blue in the Eagle's medium. The dead cells have are colored blue.

Mix suspension thoroughly by Paster pipet and apply in the chambers of hemocytometer.

Count the cells in 10 small squares of the both nets of the chamber were counted, excluding the cells, placed on lines.

If cell aggregates are detected the result was not taken into consideration, and the count has to be repeated with a re-suspended cell suspension.

Calculate the mean cell count in a square by the results obtained for three samples.

Calculate the concentration of cells in 1 ml by formula:

C=M ×25000

Where:

C=initial concentration of cells in 1 ml

M=mean value of cells in 10 small squares (3 samples).

The amount of cells applied into the well of the microplate usually has to be 2-3×10⁴ cells per 0.1 ml of growth medium.

Results

To evaluate virulicidal activity of FC-NO:GG its effect on replication of the encephalo-myocarditis virus (EMCV) in the HEp-2 cells was studied. The mixture of two equal volumes of NG extract and EMCV was incubated for 2-3 hours, and then was added to 48 hours old HEp-2 cells.

Before to start of the experiments, sterile filtration of all extracts was performed by micro-filters with pore size 0.8 and 0.2 μm (Gelman Sciences).

Test was performed as follows:

0.2 ml of the tested drug solutions was applied into each microplate's wells coated with Hep-2 and NIH 3T3 cells monolayers, except the wells of last (H) row (control). Three es for each cell culture were prepared

0.2 ml of the tested drug solutions were applied into the each well of the last row H (control—no cells) of the microplate, diluted solutions of the EMCV were prepared in doses of 50 and 100 TCD₅₀ (tissue cytotoxic dose)

Calculation of the virus' titer was performed by Koerber's formula:

log TCD₅₀=L-d (S-0.5)

Where:

L—logarithm of starting test concentration

d—a difference between log subsequent dilutions

S—sum of the proportions of the test-objects which gave a positive result (cytopathic effect)

The mixture of two equal volumes of NG extract and EMCV was incubated for 2-3 hours. 0.2ml of incubation mixture was applied in each microplate's well coated with Hep-2 layer, except the wells in the last H row (control). 3 plates were prepared.

After incubation for 48 hours the results were evaluated under a microscope, and were colored with crystal-violet (0.2 ml was added in each well).

Results of the tests were evaluated by the quantity of wells, in which the extract the cells monolayer from cytopathic action of the virus in doses of 50 and 100 TCD₅₀

TABLE 1
Virucide action of the NS-extract on EMCV (mean of 3 series of test)

| | Level of protective effect of NG | | | | | | Control + | |
|---|---|---|---|---|---|---|---|---|
| Dilution | 50 TCD₅₀ | | 100 TCD₅₀ | | Control | | NG | |
| of NG | Qty | % | Qty | % | Qty | % | Qty | % |
| 1/500 | 8 from | 66.6 | 2 from 8 | 25 | 12 | 100 | 12 | 100 |
| | 12 | | | | from | | from | |
| | | | | | 12 | | 12 | |
| 1/1000 | 0 from | 0 | 0 from 6 | 0 | | | 6 from | 100 |
| | 6 | | | | | | 6 | |
| Control | 0 from | 0 | 0 from 8 | 0 | | | | |
| EMCV | 8 | | | | | | | |

Thus, in dilution 1/500 and a low viral load (50 TCD₅₀) the NG protects the cell monolayer on 66.6%, and in higher dose of virus (100 TCD₅₀)—on 25%. In dilution 1/1000 not any virucide action was observed.

TABLE 2
Data of statistical analysis χ² for 1/500 dilution of NG
(mean of 3 series of tests)

| Dose of virus | Freedom level | χ² | Significance (p) |
|---|---|---|---|
| 50 TCD₅₀ | 1 | 6.238* | <0.05 |
| 100 TCD₅₀ | 1 | 0.633 | Not significant |

*Significant to control

Conclusions.

In conclusion, fixed combination of Nerium oleander and glycyrrhiza glabra special extracts (FC-NO:GG) in dilution 1/500 significantly exhibits virucide effect on the mice encephala-myocarditis virus in vitro.

EXAMPLE 8

Antiviral activity of a FC-NO:GG extract in patients with hepatitis B and C.

Clinical efficacy of a standardized fixed combination of special extracts (FC-NO:GG-SE), namely NatuGuardo® (S.A.V.Virex Ltd.) was studied in patients with hepatitis B and C. NatuGuard®, a liquid extract, standardized for the content of polysaccharides (0.7-1.1 mg/ml), glycyrrhizin (0.85-1.15 mg/ml) and substance's characteristics information, was given orally in a dose of 1-1.5 ml two times a day. The cases are reported from the private clinic of Dr. Husni Abu-Seir, Amman, Jordan. Four case reports were documented. Brief summary of results is presented below.

Case No. 1

Patient: Raa'd Kherfan

Age: 14 years

Hospitalized on 16/8/1997, acute Hepatitis B diagnosed. The first case in medical history treated with Natu Guard®. After 5 weeks of treatment HbsAg was negative.

Case No. 2

Patient: Asma F. Ibrahim

Age: 11 years

Hepatitis B diagnosed on 14/2/1998. After 8 weeks of treatment with Natu Guard® HbsAg was negative.

Case No. 3

Patient: Rabya Fouad

Age: 18 years

Hepatitis C diagnosed on 27/11/1999. After 6 months of treatment with Natu Guard® HCV PCR was negative.

Case No. 4

Patient: Abdel Haq Saif

Age: 35 years

Hepatitis C diagnosed on 21/3/1998. After 4 months of treatment with Natu Guard® HCV PCR was negative.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

1-4. (canceled)

5. A virucidal composition comprising a combination of Nerium oleander extract and Glycyrrhiza glabra extract.

6-17. (canceled)

18. A method for treating hepatitis B viral infection, comprising administering to a hepatitis B virus infected person in need thereof the composition according to claim 5.

19. (canceled)
